(19) United States
(12) Patent Application Publication
(10) Pub. No.: US 2011/0009352 A1
(43) Pub. Date: Jan. 13, 2011

(54) RESTORATIVE AGENT FOR ANTIBACTERIAL PEPTIDE PRODUCTION ABILITY

(75) Inventors: Fujio Suzuki, Galveston, TX (US); Makiko Kobayashi, Galveston, TX (US); Tokuichiro Utsunomiya, Yamato-shi (JP); Tsuyoshi Yoshida, Zama-shi (JP); Shohei Yoshida, Galveston, TX (US)

Correspondence Address: CONNOLLY BOVE LODGE & HUTZ, LLP, P O BOX 2207, WILMINGTON, DE, 19899, US

(73) Assignee: Minophagen Pharmaceutical Co., LTD., Tokyo (JP)

(21) Appl. No.: 12/831,559

(22) Filed: Jul. 7, 2010

Related U.S. Application Data
(60) Provisional application No. 61/224,291, filed on Jul. 9, 2009.

(51) Int. Cl. A61K 31/704; C07H 15/256; A61P 43/00

(52) U.S. Cl. 514/33; 536/18.1

(57) ABSTRACT

There is provided a medicament capable of enhancing the antimicrobial peptide production ability. The medicament contains, as an active ingredient, a compound which is glycyrrhizin or a pharmaceutically acceptable salt thereof and capable of inhibiting the production of at least one of interleukin-10 (IL-10) and chemokine CCL2. The antimicrobial peptide is preferably defensin or cathelicidin.

Priority is claimed on U.S. Patent Application No. 61/224,291, filed Jul. 9, 2009, the content of which is incorporated herein by reference.

### BACKGROUND OF THE INVENTION

1. Field of the Invention

The present invention relates to a restorative agent for antimicrobial peptide production ability which is useful for the prevention of the onset of infections.

2. Description of Related Art

Patients affected by a certain type of disease or patients who have undergone surgery often have decreased immunity against pathogenic bacteria. It is also known that these patients easily develop infections which scarcely occur in healthy individuals. In this situation, the infections not only risk the lives of the infected patients themselves, but also cause in-hospital infection, which brings prevalence of infections among patients, thus rising as a serious medical issue. Inter alia infections caused by pathogenic bacteria such as Staphylococcus aureus, Enterococcus faecalis, and Pseudomonas aeruginosa, are recently becoming a problem in particular. Usually, infections are treated by administration of antibiotic substances, and the balance of pathogenic bacteria in the body is controlled. However, the emergence of antibiotic-resistant bacteria having resistance to antibiotic substances has posed limitations on the use of antibiotic substances, so that options on the selection of effective therapeutic methods in various infections are now limited. Thus, it is strongly desired to establish a new therapeutic method.

For example, in burn patients, infection due to Pseudomonas aeruginosa is frequently observed, and it is known that even the slightest amount of Pseudomonas aeruginosa that would be absolutely problem-free in healthy individuals, can develop sepsis and the like.

The mechanism of the onset of Pseudomonas aeruginosa infection in a burn patient has been analyzed in detail using a mouse model. It is known that in a mouse suffering from a burn, the amount of murine beta-defencin 1 and 3 (hereinafter, abbreviated to MBD-1 and MBD-3, respectively), which are antimicrobial peptides that are naturally present in the skin, is markedly reduced in the skin around the burn site (see, for example, Kobayashi et al., J. Leukoc. Biol., (1354-1362), 2008), and a decrease in immunity resulting therefrom is thought to be causative of the onset of Pseudomonas aeruginosa infection. MBD-1 and MBD-3 are produced by epidermal keratinocytes, but these peptides are thought to be reduced by the action of interleukin-10 (IL-10) and chemokine CCL2, which are produced by immature myeloid cells (Gr-1⁺CD11b⁺ cells) that appear at the burnt site.

As such, even for the infections in which a decrease in the amount of production of antimicrobial peptides serves as one of the causes of crisis, a treatment by administration of antibiotic substances is predominantly used, similar to the case of other infections in related art.

### SUMMARY OF THE INVENTION

In a first aspect of the invention, a restorative agent for the antimicrobial peptide production ability including, as an active ingredient, a compound which is glycyrrhizin or a pharmaceutically acceptable salt thereof and inhibits the production of at least one of interleukin-10 and chemokine CCL2. The antimicrobial peptide is preferably defensin or cathelicidin.

In a second aspect of the invention, there is provided a protective agent against opportunistic infections including, as an active ingredient, a compound which is glycyrrhizin or a pharmaceutically acceptable salt thereof and inhibits the production of at least one of interleukin-10 and chemokine CCL2.

In a third aspect of the invention, there is provided a method for restoring the antimicrobial peptide production ability in a subject, the method including administering to the subject in need thereof an effective amount of a compound which is glycyrrhizin or a pharmaceutically acceptable salt thereof and inhibits the production of at least one of interleukin-10 and chemokine CCL2.

In a fourth aspect of the invention, there is provided a method for protecting against opportunistic infections in a subject, the method including administering to the subject in need thereof an effective amount of a compound which is glycyrrhizin or a pharmaceutically acceptable salt thereof and inhibits the production of at least one of interleukin-10 and chemokine CCL2.

### BRIEF DESCRIPTION OF THE DRAWINGS

FIG. 1 is a graph showing the survival rate of mice in Example 1 and Comparative Example 1.

FIG. 2 is a graph showing the survival rate of mice in Reference Examples 1 to

FIG. 3 is a graph showing the survival rate of mice in Reference Examples 4 to 6.

FIG. 4 is a graph showing the amount of Pseudomonas aeruginosa in a mouse blood specimen and a mouse spleen homogenate specimen obtained in Example 2 and Comparative Example 2.

FIG. 5 is a graph showing the amount of MBD-1 in a mouse skin tissue homogenate liquid obtained in Example 3 and Comparative Example 3.

FIG. 6 is a graph showing the amount of MBD-1 in the supernatant of a culture fluid of epidermal keratinocytes and/or Gr-1⁺CD11b⁺ cells of mouse obtained in Example 4, Comparative Example 4 and Reference Examples 7 to 9.

FIG. 7 is a graph showing the amount of MBD-3 in the supernatant of a culture fluid of LPS-treated epidermal keratinocytes and/or Gr-1⁺CD11b⁺ cells of mouse obtained in Example 5, Comparative Example 5 and Reference Examples 10 to 12.

FIG. 8 is a graph showing the amount of MBD-1 in the supernatant of a culture fluid of epidermal keratinocytes and Gr-1⁺CD11b⁺ cells of mouse, or in the supernatant of a culture fluid of epidermal keratinocytes of mouse, obtained in Examples 6 to 9, Comparative Example 6 and Reference Examples 7 and 8.

FIG. 9 is a graph showing the amounts of IL-4, IL-13, IL-10 and CCL2 in the supernatant of a culture fluid of mouse Gr-1⁺CD11b⁺ cells obtained in Reference Examples 13 to 15.

FIG. 10 is a graph showing the amount of MBD-1 in the supernatant of a culture fluid of mouse epidermal keratinocytes obtained in Reference Examples 13 to 17.

FIG. 11 is a graph showing the amount of MBD-1 in the supernatant of a culture fluid of mouse epidermal keratinocytes obtained in Reference Example 17 and Reference Examples 18 to 20.

FIG. 12 is a graph showing the amount of CCL2 in the supernatant of a culture fluid of mouse Gr-1⁺CD11b⁺ cells obtained in Example 10 and Comparative Example 7.

FIG. 13 is a graph showing the amount of IL-10 in the supernatant of a culture fluid of mouse Gr-1⁺CD11b⁺ cells obtained in Example 10 and Comparative Example 7.

### DETAILED DESCRIPTION OF THE INVENTION

As described above, administration of antibiotic substances has a problem that there is a possibility of producing new antibiotic-resistant bacteria. The risk as such is particularly high with, for example, Pseudomonas aeruginosa having high ability for acquisition of new drug resistance. In most of the infections, there has been a problem that there is no effective therapeutic method that will replace the administration of antibiotic substances. Accordingly, it is strongly desired to develop a highly safe and highly effective prophylactic method for the onset of infection, which would replace the administration of antibiotic substances. For example, a prophylactic method will be considered promising for the onset of infections, if the immunity of a patient himself/herself to pathogenic bacteria can be restored to a level comparable to the level of immunity in a healthy individual. Furthermore, concerning infections in which a decrease in the amount of production of antimicrobial peptides is causative of the onset, it is considered important to increase the amount of production of antimicrobial peptides to a level comparable to the level in a healthy individual. However, such a prophylactic method for the onset of infections has not been known yet.

The present invention was made under such circumstances, and it is an object of the invention to provide a medicament capable of increasing the amount of production of antimicrobial peptides.

The present inventors conducted a thorough investigation to solve the technical problems described above, and as a result, they found that glycyrrhizin, which is a therapeutic drug for hepatic diseases or allergic diseases and has been recognized for its safety, absolutely unexpectedly enhances the amount of production of MBD-1 and MBD-3 in mice, thus completing the invention.

According to the invention, the amount of production of antimicrobial peptides can be enhanced, and the onset of infections can be prevented. Furthermore, a method for preventing the onset of infections, which method has a low risk of producing new antimicrobial-resistant bacteria and is highly safe, can be provided.

The restorative agent for antimicrobial peptide production ability (hereinafter, simply referred to as restorative agent) of the invention contains, as an active ingredient, a compound which is glycyrrhizin or a pharmaceutically acceptable salt thereof and inhibits the production of at least one of interleukin-10 (hereinafter, abbreviated to IL-10) and chemokine CCL2 (hereinafter, abbreviated to CCL2).

The restorative agent of the invention is thought to be capable of restoring the antimicrobial peptide production ability by acting on cells that produce at least one of IL-10 and CCL2 and inhibiting the production ability for these peptides.

The antimicrobial peptides for which the restorative agent of the invention can restore the production ability, are peptides whose production is inhibited by IL-10 or CCL2. Among them, defensins such as α-defensin and β-defensin, and cathelicidin are suitable. Suitable examples of β-defensin include human β-defensins (HBDs) such as human β-defensin-1(HBD-1), human β-defensin-2 (HBD-2), human β-defensin-3 (HBD-3) and human β-defensin-4 (HBD-4). Suitable examples of a-defensin include human neutrophilic defensins (HNPs) such as human neutrophilic defensin-1 (HNP-1), human neutrophilic defensin-2 (HNP-2) and human neutrophilic defensin-3 (HNP-3). Suitable examples of cathelicidin include cathelicidin antimicrobial peptide-18 (CAP-18).

Glycyrrhizin or a pharmaceutically acceptable salt thereof can be obtained through, for example, extraction from licorice, but a commercially available product may also be used. A suitable example of the commercially available product is a product manufactured by Minophagen Pharmaceutical Co., Ltd.

Examples of the pharmaceutically acceptable salt of glycyrrhizin include those salts obtainable by allowing glycyrrhizin and an inorganic or organic base to react at a certain molar ratio. Specific preferred examples thereof include ammonium salts such as glycyrrhizin monoammonium salt and glycyrrhizin diammonium salt; alkali metal salts such as glycyrrhizin monosodium salt, glycyrrhizin disodium salt, glycyrrhizin monopotassium salt and glycyrrhizin dipotassium salt; glycyrrhizin choline salt; glycyrrhizin calcium salt; glycyrrhizin magnesium salt; glycyrrhizin aluminum salt; and the like. Among these, glycyrrhizin monoammonium salt is particularly preferred.

Glycyrrhizin or a pharmaceutically acceptable salt thereof may be used alone, or two or more kinds may be used together. In the case of using two or more kinds together, the combination and the ratio may be appropriately adjusted according to the purpose.

The dosage form of the restorative agent of the invention is not particularly limited and may be appropriately selected from oral preparations such as tablets, powders, granules, capsules, fine granules and liquids (solutions); parenteral preparations such as inhalants, suppositories and injectable preparations; and the like, according to the purpose. Restorative agents in the form of these dosage forms can all be produced by known methods.

In the case of formulating the agent in the form of an oral preparation or the like, excipients, lubicants, plasticizers, surfactants, binders, disintegrants, wetting agents, stabilizers, corrigents, colorants, flavors, buffering agents and the like that are generally used in the production of these preparations may be incorporated into the oral preparation.

Examples of the excipient include lactose, glucose, D-mannitol, fructose, dextrin, starch, table salt, sodium hydrogen carbonate, calcium carbonate, sodium alginate, ethylcellulose, sodium carboxymethylcellulose, hydroxypropylcellulose, silicic anhydride, kaolin, and the like.

Examples of the lubricant include magnesium stearate, calcium stearate, stearic acid, talc, corn starch, macrogol, and the like.

Examples of the plasticizer include polyethylene glycol, propylene glycol, glycerins, triacetin, medium-chain fatty acid triglyceride, acetylglycerin fatty acid esters, triethyl citrate, and the like.

Examples of the binder include gelatin, gum arabic, cellulose esters, polyvinylpyrrolidone, starch syrup, an extract of licorice, tragacanth, simple syrup, gelatin, and the like.

Examples of the disintegrant include starch, agar, carmellose calcium, carmellose, crystalline cellulose, and the like.

Examples of the wetting agent include gum arabic, polyvinylpyrrolidone, methylcellulose, carmellose sodium, hydroxypropylcellulose, and the like.

Examples of the corrigent include sugar, honey, saccharin sodium, mint, eucalyptus oil, cinnamon oil, and the like.

Examples of the colorant include iron oxide, β-carotene, chlorophyll, water-soluble edible tar dyes, and the like.

Examples of the flavor include lemon oil, orange oil, dl- or 1-menthol, and the like.

In the case of formulating the agent in the form of a parenteral preparation such as an inhalant or an injectable preparation, examples of solvents that can be used include distilled water for injection, a sterilized non-aqueous solvent, a suspension agent, and the like. Preferred examples of the base material of the non-aqueous solvent or suspension agent include propylene glycol, polyethylene glycol, glycerin, olive oil, corn oil, ethyl oleate, and the like.

The restorative agent of the invention may have, if necessary, pharmaceutically acceptable optional components other than the components mentioned above incorporated therein, so long as the components do not obstruct the effects of the invention.

Examples of the optional components include a buffering agent, a preservative, an antioxidant, and the like.

The method of administration of the restorative agent of the invention may involve any of oral administration or parenteral administration.

The dosage of the restorative agent may adequately vary depending on the age, symptoms and the like of the patient, but in the case of oral administration, the daily dose for an adult is usually, in terms of the amount of glycyrrhizin or a pharmaceutically acceptable salt thereof, preferably 50 to 3000 mg/person, and more preferably 300 to 2500 mg/person, while in the case of parenteral administration, the daily dose for an adult is usually, in terms of the amount of glycyrrhizin or a pharmaceutically acceptable salt thereof, preferably 25 to 2500 mg/person, and more preferably 150 to 2000 mg/person.

The restorative agent of the invention is administered such that a predetermined amount is administered once or in several divided portions a day.

Glycyrrhizin and pharmaceutically acceptable salts thereof have a long record of use as therapeutic drugs for hepatic diseases and allergic diseases, and have a reputation for their high safety. Since the restorative agent of the invention is formed from glycyrrhizin or a pharmaceutically acceptable salt thereof, the restorative agent is highly safe. Furthermore, the restorative agent has an action of reinforcing a function that is originally possessed by a living body, which is related to the restoration of the antimicrobial peptide production ability, and thus the restorative agent has a low risk of producing new antimicrobial-resistant bacteria. As such, the restorative agent of the invention provides a highly safe method for preventing the onset of infections.

The restorative agent of the invention can be suitably applied to burn patients, but so long as the restorative agent restores the antimicrobial peptide production ability by inhibiting the production of at least one of IL-10 and CCL2, the subject in need thereof is not limited to burn patients, and the subject in need thereof can be extended to all of those patients who need the prevention of the onset of infections.

### EXAMPLES

Hereinafter, the invention will be further described in detail with reference to specific Examples. However, the invention is not by any means intended to be limited to the following Examples.

### Comparison of Survival Rate of Pseudomonas Aeruginosa-Administered Mice

### Example 1

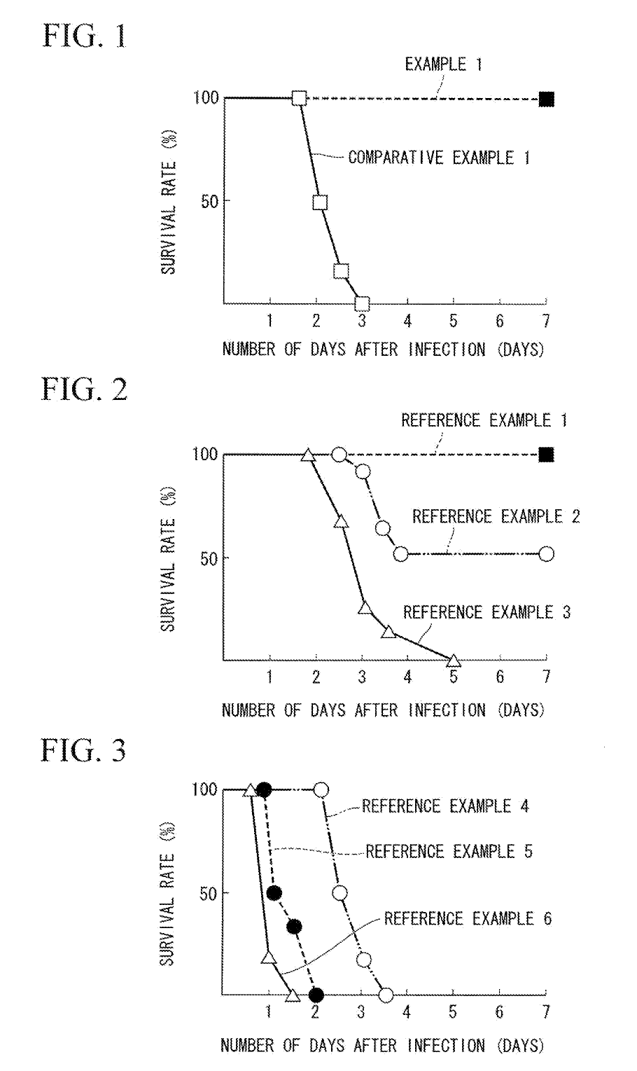

A normal mouse was given a burn injury on the dorsal side, and Pseudomonas aeruginosa was applied to the burn site in an amount of 100 CFU (Colony-Forming Units). Subsequently, glycyrrhizin was intraperitoneally administered to the mouse at 2 hours, 24 hours and 72 hours after the occurrence of burn injury, each time in an amount of 10 mg per kilogram of body weight of the mouse (an amount of 10 mg/kg), and it was checked whether the mouse was alive or dead. The same operation was carried out on 10 mice, and the survival rate of the mice was checked. The results are shown in FIG. 1. The horizontal axis of the graph of FIG. 1 represents the number of days (days) after the Pseudomonas aeruginosa infection.

### Comparative Example 1

The experiment was carried out in the same manner as in Example 1, except that physiological saline was intraperitoneally administered instead of glycyrrhizin, and the survival rate of the mice was checked. The results are shown in FIG. 1.

As it is obvious in FIG. 1, none of the individuals died up to the 7^{th} day among the mice administered with glycyrrhizin (Example 1), and the survival rate after 2 days and on was markedly high as compared with the mice which were not administered with glycyrrhizin (Comparative Example 1).

### Reference Examples 1 to 3

Normal mice were each infected on their dorsal side with Pseudomonas aeruginosa in an amount of 10⁶ CFU (Reference Example 1), 10⁷ CFU (Reference Example 2) or 10⁸ CFU (Reference Example 3) per mouse, and it was checked whether the mice were alive or dead. The same operation was carried out on 10 mice per group, and the survival rate of the mice was checked. The results are shown in FIG. 2. The horizontal axis of the graph of FIG. 2 represents the number of days (days) after the infection (application of Pseudomonas aeruginosa).

As it is obvious from FIG. 2, a larger dose of Pseudomonas aeruginosa resulted in a lower survival rate of mice.

### Reference Examples 4 to 6

Normal mice were each given a burn injury on their dorsal side, and were intradermally infected at the burn site with Pseudomonas aeruginosa in an amount of 50 CFU (Reference Example 4), 10³ CFU (Reference Example 5) or 10⁴ CFU (Reference Example 6) per mouse, and it was checked whether the mice were alive or dead. The same operation was carried out on 10 mice per group, and the survival rate of the mice was checked. The results are shown in FIG. 3. The horizontal axis of the graph of FIG. 3 represents the lapse of time (days) after the infection of Pseudomonas aeruginosa.

As it is obvious from FIG. 3, the survival rate of the mice was lowered such that Pseudomonas aeruginosa infection occurred many times with a smaller dose of Pseudomonas aeruginosa than that of Reference Examples 1 to 3.

### Comparison of Amount of Pseudomonas aeruginosa in Test Specimen of Pseudomonas aeruginosa-Infected Mouse

### Example 2

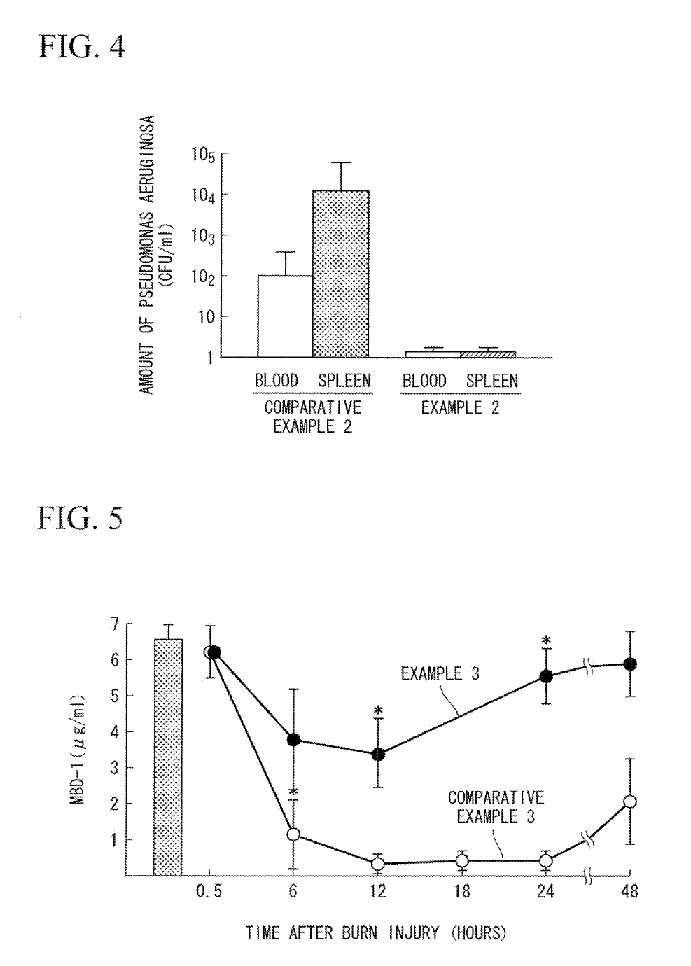

A normal mouse was given a burn injury on its dorsal side, and was infected at the burn site with Pseudomonas aeruginosa in an amount of 100 CFU. Subsequently, glycyrrhizin was intraperitoneally administered to the mouse at 2 hours and 12 hours after the infection with Pseudomonas aeruginosa, each time in an amount of 10 mg per kilogram of body weight of the mouse (an amount of 10 mg/kg). Subsequently, after 48 hours from the infection with Pseudomonas aeruginosa, blood and spleen were collected from the mouse, and the amounts of Pseudomonas aeruginosa in the blood specimen and the spleen homogenate specimen were measured by a colony-counting method. The results are shown in FIG. 4. The vertical axis of the graph of FIG. 4 represents the amount of Pseudomonas aeruginosa in 1 ml of the specimen (amount of CFU).

### Comparative Example 2

The experiment was carried out in the same manner as in Example 2, except that physiological saline was intraperitoneally administered instead of glycyrrhizin, and the amounts of Pseudomonas aeruginosa in the blood specimen and the spleen homogenate specimen were measured. The results are shown in FIG. 4.

As it is obvious from FIG. 4, the mouse administered with glycyrrhizin (Example 2) had a markedly reduced amount of Pseudomonas aeruginosa in both the blood specimen and the spleen homogenate specimen, as compared with the mouse which was not administered (Comparative Example 2).

### Comparison of Amount of MBD-1 in Skin Tissue

### Example 3

A normal mouse was given a burn injury on its dorsal side, and was intraperitoneally administered with glycyrrhizin in an amount of 10 mg per kg of body weight of the mouse (an amount of 10 mg/kg), 2 hours after the occurrence of burn injury. The skin tissue was collected from the surroundings of the burn site after 0.5 hours, 6 hours, 12 hours, 18 hours and 24 hours from the occurrence of burn injury, and the collected skin tissues were homogenized. The amount of MBD-1 in the homogenate liquid was measured by ELISA. The results are shown in FIG. 5. The horizontal axis of the graph of FIG. 5 represents the time (hours) after the occurrence of burn injury.

### Comparative Example 3

The experiment was carried out in the same manner as in Example 3, except that physiological saline was intraperitoneally administered instead of glycyrrhizin, and the amount of MBD-1 in the homogenate liquid was measured. The results are shown in FIG. 5.

As it is obvious from FIG. 5, the amount of MBD-1 was larger in the mouse administered with glycyrrhizin (Example 3) than in the mouse which was not administered (Comparative Example 3).

### Comparison of Amount of MBD-1 in Cultured Cells

### Example 4

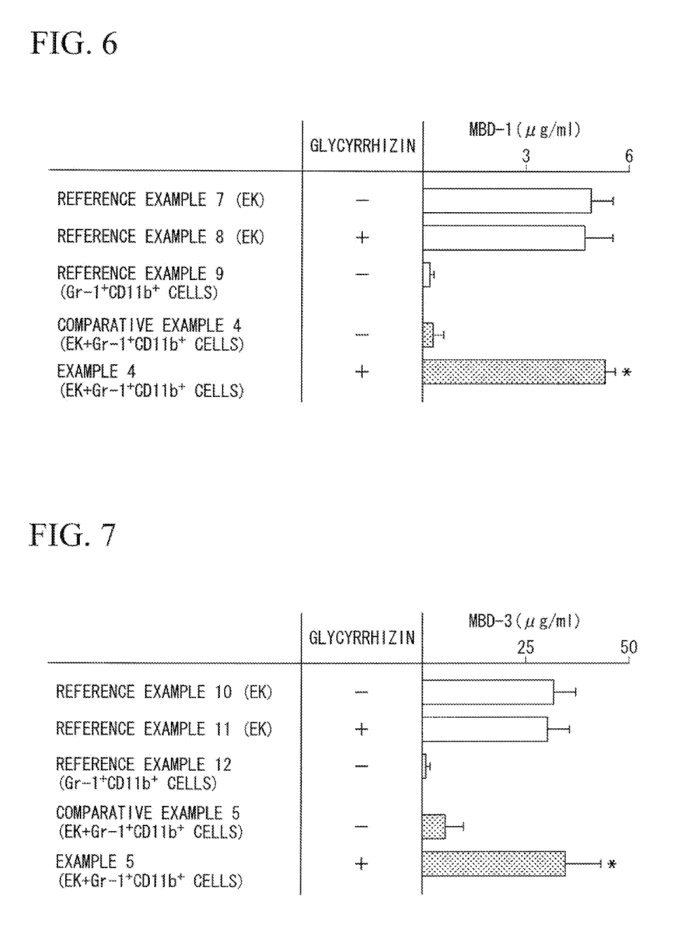

Epidermal keratinocytes (EK) were collected from the skin of a normal mouse, and a cell solution was prepared to a concentration of 2×10⁶ cells/ml using RPMI1640 medium supplemented with 10% inactivated fetal bovine serum. Furthermore, a normal mouse was given a burn injury on its dorsal side, and after 12 hours, Gr-1⁺CD11b⁺ cells were collected from the skin tissues around the burn site. A cell solution of the collected cells was prepared to a concentration of 5×10⁵ cells/ml using RPMI1640 medium supplemented with 10% inactivated fetal bovine serum. In the co-presence of glycyrrhizin in an amount of 10 μg/ml, the epidermal keratinocytes and the Gr-1⁺CD11b⁺ cells were cultured in a same Transwell at 37° C. After 48 hours from the initiation of culture, the amount of MBD-1 in the supernatant of the culture fluid was measured by ELISA. The results are shown in FIG. 6.

### Comparative Example 4

The experiment was carried out in the same manner as in Example 4, except that glycyrrhizin was not allowed to be co-present, and the amount of MBD-1 in the supernatant of the culture fluid was measured. The results are shown in FIG. 6.

### Reference Example 7

The experiment was carried out in the same manner as in Example 4, except that only epidermal keratinocytes (2×10⁶ cells/ml) were cultured without allowing glycyrrhizin to be co-present, and the amount of MBD-1 in the supernatant of the culture fluid was measured. The results are shown in FIG. 6.

### Reference Example 8

The experiment was carried out in the same manner as in Example 4, except that only epidermal keratinocytes (2×10⁶ cells/ml) were cultured in the co-presence of glycyrrhizin in an amount of 10 μg/ml, and the amount of MBD-1 in the supernatant of the culture fluid was measured. The results are shown in FIG. 6.

### Reference Example 9

The experiment was carried out in the same manner as in Example 4, except that only Gr-1⁺CD11b⁺ cells (5×10⁵ cells/ml) were cultured without allowing glycyrrhizin to be co-present, and the amount of MBD-1 in the supernatant of the culture fluid was measured. The results are shown in FIG. 6.

As it is obvious from FIG. 6, the amount of MBD-1 was larger in the Example 4 where glycyrrhizin was made to be co-present, as compared to the Comparative Example 4 where glycyrrhizin was not co-present. Meanwhile, glycyrrhizin did not have an MBD-1 production ability promoting effect against the epidermal keratinocytes (Reference Examples 7 and 8). However, the Gr-1⁺CD11b⁺ cells had an MBD-1 production ability inhibiting effect against the epidermal keratinocytes, but glycyrrhizin reduced this inhibiting effect. Thus, it could be confirmed that glycyrrhizin acts as a restorative agent for MBD-1 production ability.

### Comparison of Amount of MBD-3 in Cultured Cells

### Example 5

Epidermal keratinocytes (EK) were collected from the skin of a normal mouse, and a cell solution was prepared to a concentration of 2×10⁶ cells/ml using RPMI1640 medium supplemented with 10% inactivated fetal bovine serum. In order to induce MBD-3, the cells were treated with lipopolysaccharides (LPS) in an amount of 1 μg/ml for 6 hours. Furthermore, a normal mouse was given a burn injury on its dorsal side, and after 12 hours, Gr-1⁺CD11b⁺ cells were collected from the skin tissues around the burn site. A cell solution of the collected cells was prepared to a concentration of 5×10⁵ cells/ml using RPMI1640 medium supplemented with 10% inactivated fetal bovine serum. In the co-presence of glycyrrhizin in an amount of 10 μg/ml, the LPS-treated epidermal keratinocytes and the Gr-1⁺CD11b⁺ cells were cultured in a same Transwell at 37° C. After 48 hours from the initiation of culture, the amount of MBD-3 in the supernatant of the culture fluid was measured by ELISA. The results are shown in FIG. 7.

### Comparative Example 5

The experiment was carried out in the same manner as in Example 5, except that glycyrrhizin was not allowed to be co-present, and the amount of MBD-3 in the supernatant of the culture fluid was measured. The results are shown in FIG. 7.

### Reference Example 10

The experiment was carried out in the same manner as in Example 5, except that only LPS-treated epidermal keratinocytes (2×10⁶ cells/ml) were cultured without allowing glycyrrhizin to be co-present, and the amount of MBD-3 in the supernatant of the culture fluid was measured. The results are shown in FIG. 7.

### Reference Example 11

The experiment was carried out in the same manner as in Example 5, except that only LPS-treated epidermal keratinocytes (2×10⁶ cells/ml) were cultured in the co-presence of glycyrrhizin in an amount of 10 μg/ml, and the amount of MBD-3 in the supernatant of the culture fluid was measured. The results are shown in FIG. 7.

### Reference Example 12

The experiment was carried out in the same manner as in Example 5, except that only Gr-1⁺CD11b⁺ cells (5×10⁵ cells/ml) were cultured without allowing glycyrrhizin to be co-present, and the amount of MBD-3 in the supernatant of the culture fluid was measured. The results are shown in FIG. 7.

As it is obvious from FIG. 7, the amount of MBD-3 was larger in the Example 5 where glycyrrhizin was made to be co-present, as compared to the Comparative Example 5 where glycyrrhizin was not co-present. Meanwhile, glycyrrhizin did not have an MBD-3 production ability promoting effect against the LPS-treated epidermal keratinocytes (Reference Examples 10 and 11). However, the Gr-1⁺CD11b⁺ cells had an MBD-3 production ability inhibiting effect against the LPS-treated epidermal keratinocytes, but glycyrrhizin reduced this inhibiting effect. Thus, it could be confirmed that glycyrrhizin acts as a restorative agent for MBD-3 production ability.

### Verification of Quantity-Dependency of Glycyrrhizin on MBD-1 Production Ability Restoration

### Examples 6 to 9

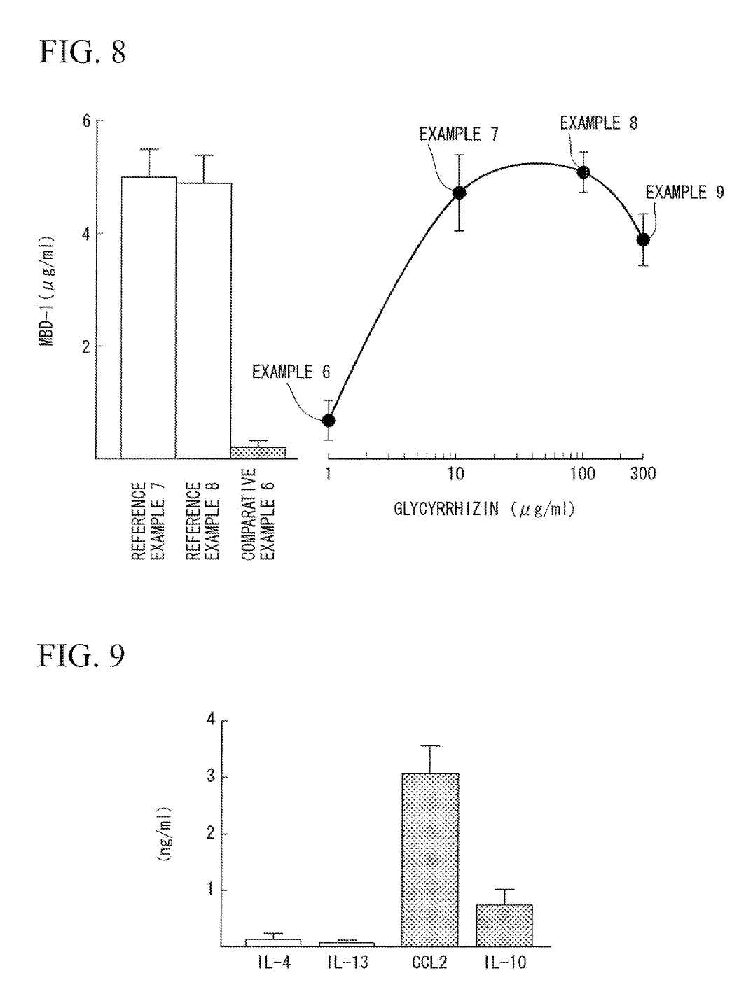

Epidermal keratinocytes (2×10⁶ cells/ml) were collected from the skin of a normal mouse. Furthermore, a normal mouse was given a burn injury on its dorsal side, and after 12 hours, Gr-1⁺CD11b⁺ cells (5×10⁵ cells/ml) were collected from the skin tissues around the burn site. Using the medium for keratinocyte culture as a medium, and making glycyrrhizin to be co-present in an amount of 1 μg/ml (Example 6), 10 μg/ml (Example 7), 100 μg/ml (Example 8) or 300 μg/ml (Example 9), the epidermal keratinocytes and the Gr-1⁺CD11b⁺ cells were cultured in a same Transwell at 37° C. The amount of MBD-1 in the supernatant of the culture fluid after 48 hours from the initiation of culture was measured by ELISA. The results are shown in the semilogarithmic graph of FIG. 8.

### Comparative Example 6

The experiment was carried out in the same manner as in Examples 6 to 9, except that glycyrrhizin was not made to be co-present, and the amount of MBD-1 in the supernatant of the culture fluid was measured. The results are shown in the bar graph of FIG. 8. FIG. 8 also shows the results of the Reference Examples 7 and 8. Furthermore, the two graphs share the vertical axis (the amount of MBD-1).

As it is obvious from FIG. 8, it could be confirmed that when glycyrrhizin was present in an amount of 10 μg/ml (Example 7), 100 μg/ml (Example 8) and 300 μg/ml (Example 9), the MBD-1 production ability restoring effect was particularly excellent.

### Verification of MBD-1 Production Ability Inhibitory Factor of Epidermal Keratinocytes (1)

### Reference Examples 13 to 15

A normal mouse was given a burn injury on its dorsal side, and after 12 hours, Gr-1⁺CD11b⁺ cells (5×10⁵ cells/ml) were collected from the skin tissues around the burn site. A cell solution of the collected cells was prepared to a concentration of 5×10⁵ cells/ml using RPMI1640 medium supplemented with 10% inactivated fetal bovine serum. The supernatant was recovered after 48 hours from the initiation of culture, and the amounts of IL-4, IL-13, IL-10 and CCL2 in the supernatant specimen were measured by ELISA. The results are shown in FIG. 9.

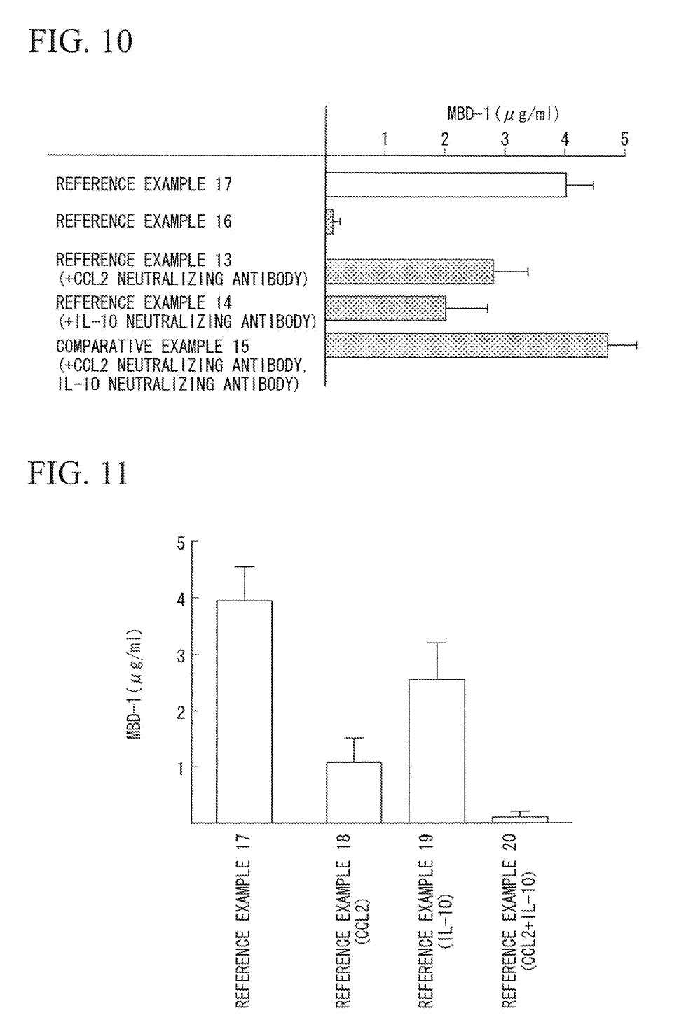

Subsequently, the supernatant of the culture fluid was treated with a CCL2 neutralizing antibody (Reference Example 13), with an IL-10 neutralizing antibody (Reference Example 14), or with a CCL2 neutralizing antibody and an IL-10 neutralizing antibody, each in an amount of 2.5 μg/ml, at 37° C. for 1 hour. The supernatant of the treated culture fluid was added to a final concentration equivalent to 15 volume % of the medium. This medium was used to culture the epidermal keratinocytes (2×10⁶ cells/ml) separated from the skin tissue of a normal mouse, at 37° C. After 48 hours from the initiation of culture, the supernatant was recovered, and the amount of MBD-1 in the supernatant specimen was measured by ELISA. The results are shown in FIG. 10.

### Reference Example 16

The experiment was carried out in the same manner as in Reference Examples 13 to 15, except that neither the CCL2 neutralizing antibody nor the IL-10 neutralizing antibody was used, and the amount of MBD-1 in the supernatant of the culture fluid was measured. The results are shown in FIG. 10.

### Reference Example 17

An isotype control antibody to a neutralizing antibody was used to culture the epidermal keratinocytes (2×10⁶ cells/ml) collected from the skin of a normal mouse, at 37° C. The amount of MBD-1 in the supernatant of the culture fluid after 48 hours from the initiation of culture was measured by ELISA. The results are shown in FIG. 10.

As it is obvious from FIG. 10, it was confirmed that when the CCL2 neutralizing antibody and the IL-10 neutralizing antibody were added together, the MBD-1 production ability of the epidermal keratinocytes was inhibited the most (Reference Example 16). Furthermore, it was confirmed that when at least one of the CCL2 neutralizing antibody and the IL-10 neutralizing antibody was added, the MBD-1 production ability of the epidermal keratinocytes was restored (Reference Examples 13 to 15). Furthermore, it was confirmed that the MBD-1 production ability restoring effect was higher when both of the CCL2 neutralizing antibody and the IL-10 neutralizing antibody were added (Reference Example 15), as compared to the case of adding only one peptide (Reference Examples 13 and 14).

From the above results, it was shown that the CCL2 and IL-10 derived from Gr-1⁺CD11b⁺ cells inhibited the MBD-1 production ability restoring effect of the epidermal keratinocytes.

### Verification of MBD-1 Production Ability Inhibitory Factor of Epidermal Keratinocytes (2)

### Reference Examples 18 to 20

Epidermal keratinocytes (2×10⁶ cells/ml) collected from the skin of a normal mouse were cultured at 37° C. in the co-presence of CCL2 (5 ng/ml) (Reference Example 18), IL-10 (1 ng/ml) (Reference Example 19), or CCL2 (5 ng/ml) and IL-10 (1 ng/ml) (Reference Example 20), which had been produced by a known recombinant DNA technology using a recombinant. The amount of MBD-1 in the supernatant of the culture fluid after 48 hours from the initiation of culture was measured by ELISA. The results are shown in FIG. 11. FIG. 11 also shows the results of the Reference Example 17.

As it is obvious from FIG. 11, it was confirmed that the MBD-1 production ability of the epidermal keratinocytes was inhibited by adding at least one of CCL2 and IL-10 (Reference Examples 18 to 20). It was confirmed that the MBD-1 production ability inhibiting effect was higher when CCL2 and IL-10 were both added (Reference Example 20), as compared to the case of adding only one of the peptides (Reference Examples 18 and 19).

From the results above, it was shown that the MBD-1 production ability of the epidermal keratinocytes was inhibited by the CCL2 and IL-10 derived from Gr-1⁺CD11b⁺ cells.

### Verification of CCL2 and IL-10 Production Inhibiting Effect of Glycyrrhizin

### Example 10

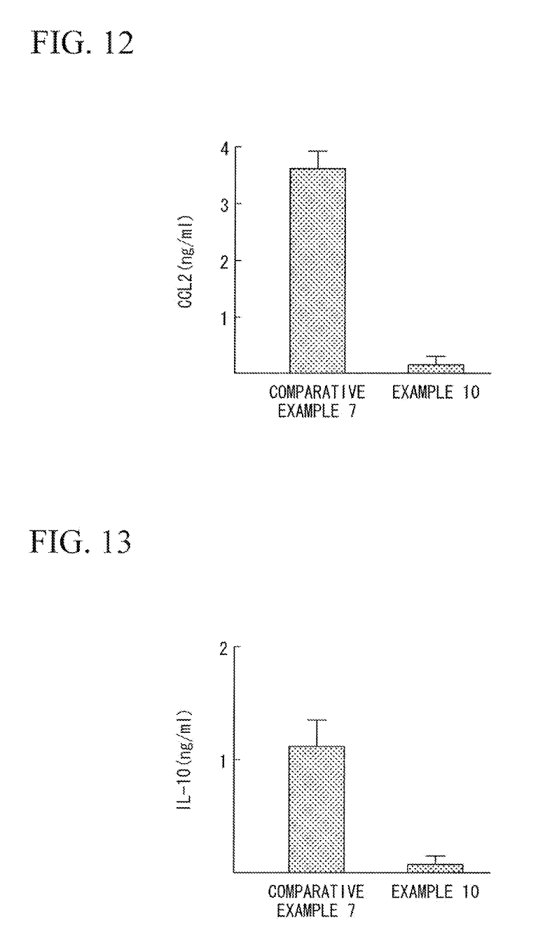

A normal mouse was given a burn injury on its dorsal side, and after 12 hours, Gr-1⁺CD11b⁺ cells (2×10⁶ cells/ml) were collected from the skin tissue around the burn site. Using the keratinocyte culture medium as a medium, the Gr-1⁺CD11b⁺ cells were cultured in a 96-well plate at 37° C. in the co-present of glycyrrhizin in an amount of 10 μg/ml. After 48 hours from the initiation of culture, the amounts of CCL2 and IL-10 in the supernatant of the culture fluid were measured by ELISA. The results are shown in FIGS. 12 and 13. FIG. 12 shows the amount of CCL2, and FIG. 13 shows the amount of IL-10.

### Comparative Example 7

The experiment was carried out in the same manner as in Example 10, except that glycyrrhizin was not made to be co-present, and the amounts of CCL2 and IL-10 in the supernatant of the culture fluid were measured. The results are shown in FIGS. 12 and 13.

As it is obvious from FIGS. 12 and 13, it could be confirmed that glycyrrhizin inhibited both the CCL2 and IL-10 production ability of Gr-1⁺CD11b⁺ cells.

The invention can be used in the prevention of infections in burn patients.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A restorative agent for antimicrobial peptide production ability comprising glycyrrhizin or a pharmaceutically acceptable salt thereof as an active ingredient.

2. The restorative agent according to claim 1, wherein the antimicrobial peptide is defensin or cathelicidin.

3. The restorative agent according to claim 1, which is in a form selected from tablets, a powder, granules, capsules, fine granules, a liquid, an inhalant, a suppository and an injectable preparation.

4. A protective agent for opportunistic infection comprising glycyrrhizin or a pharmaceutically acceptable salt thereof as an active ingredient.

5. A method for restoring the antimicrobial peptide production ability in a subject comprising:
administering to the subject in need thereof an effective amount of glycyrrhizin or a pharmaceutically acceptable salt thereof.

6. The method according to claim 5, wherein the glycyrrhizin or a pharmaceutically acceptable salt thereof is orally administered to the subject in need thereof.

7. The method according to claim 6, wherein the daily dosage is 50 to 3000 mg for the subject in need thereof, in terms of the amount of the glycyrrhizin or a pharmaceutically acceptable salt thereof.

8. The method according to claim 7, wherein the administration is carried out once or in several divided portions a day.

9. The method according to claim 5, wherein the glycyrrhizin or a pharmaceutically acceptable salt thereof is parenterally administered to the subject in need thereof.

10. The method according to claim 9, wherein the daily dosage is 25 to 2500 mg for the subject in need thereof, in terms of the amount of the glycyrrhizin or a pharmaceutically acceptable salt thereof.

11. The method according to claim 10, wherein the administration is carried out once or in several divided portions a day.

12. A method for protecting against opportunistic infection in a subject comprising:
administering to the subject in need thereof an effective amount of glycyrrhizin or a pharmaceutically acceptable salt thereof.
